(19) United States
(12) Patent Application Publication
(10) Pub. No.: US 2019/0183672 A1
(43) Pub. Date: Jun. 20, 2019

(54) MEDICAL COOLING PAD

(71) Applicant: ANJA ELLERBRAKE-SADLER, KOELN (DE)

(72) Inventor: ANJA ELLERBRAKE-SADLER, KOELN (DE)

(21) Appl. No.: 16/322,120

(22) PCT Filed: Aug. 3, 2016
(86) PCT No.: PCT/EP2016/068581

§ 371 Date: Jan. 31, 2019

(51) Int. Cl. A61F 7/02; A61F 13/02

(52) U.S. Cl. CPC A61F 7/02; A61F 2007/0052; A61F 2007/0219; A61F 13/0226

(57) ABSTRACT

A medical cooling pad for cooling skin includes a distal flexible pad support, a flexible cooling gel layer arranged on a proximal side of the distal flexible pad support, and a medical wound dressing arranged on a proximal side of the flexible cooling gel layer. The medical wound dressing has an area which is smaller than an area of the flexible cooling gel layer.

### CROSS REFERENCE TO PRIOR APPLICATIONS

This application is a U.S. National Phase application under 35 U.S.C. § 371 of International Application No. PCT/EP2016/068581, filed on Aug. 3, 2016. The International Application was published in German on Feb. 8, 2018 as WO 2018/024329 A1 under PCT Article 21(2).

### FIELD

The present invention relates to a medical cooling pad for cooling human skin or an injury on and/or under the skin.

### BACKGROUND

State-of-the-art medical cooling pads consist of a cushion with a liquid, gel-like, sand-like or earth-like filling that has a relatively high heat capacity and is applied to the skin in a cooled state and is fixed by a separate bandaging. It is cumbersome to apply the cushion-like cooling pad to the skin.

DE 102 60 872 A1 and DE 10 2010 038 312 describe cooling pads which are self-adhesive so that no further means and working steps are necessary for fixation on the skin after the application thereof on the skin.

### SUMMARY

An aspect of the present invention is to provide a medical cooling pad that can also be used for external skin injuries.

In an embodiment, the present invention provides a medical cooling pad for cooling skin which includes a distal flexible pad support, a flexible cooling gel layer arranged on a proximal side of the distal flexible pad support, and a medical wound dressing arranged on a proximal side of the flexible cooling gel layer. The medical wound dressing has an area which is smaller than an area of the flexible cooling gel layer.

### BRIEF DESCRIPTION OF THE DRAWINGS

The present invention is described in greater detail below on the basis of embodiments and of the drawings in which:

FIG. 1 shows a longitudinal section of a medical cooling pad packed in a protective bag;

FIG. 2 shows the medical cooling pad without a protective bag;

FIG. 3 shows the medical cooling pad after removing the cooling pad protective strip;

FIG. 4 shows the medical cooling pad after removing the wound dressing protective strips;

FIG. 5 shows the medical cooling pad of FIG. 4 applied to the skin;

FIG. 6 shows the medical cooling pad of FIG. 5 after the pad support has been removed from the skin together with the cooling gel layer so that the adhesive tape with the wound dressing remains attached to the skin; and

FIG. 7 shows the proximal side of the cooling pad of FIG. 3 in a plan view.

### DETAILED DESCRIPTION

The medical cooling pad for cooling the skin comprises a distal flexible pad support with a flexible cooling gel layer applied to its proximal side. The term “proximal” generally refers to the side facing the skin after application, whereas the term “distal” refers to the side facing away from the skin after application. Both the pad support and the cooling gel layer are flexible to the extent that the cooling pad can adapt to the spatial shape of the skin surface. A cooling gel layer is any type of coating that allows a passive or active cooling of the skin. A cooling gel layer is not necessarily a gel in the narrower sense, but any substance that can have a cooling effect and has a certain stability, i.e., is non-fluidic at body temperature.

“Passive cooling” is defined as cooling by a pre-cooled layer, for example, in a refrigerator, of a cooling gel having a relatively high heat capacity. Passive cooling can, however, also be defined by cooling via storing a liquid in the cooling gel layer, whereby the liquid evaporates after being applied to the skin and thus produces evaporative cooling. Suitable liquids are water and/or volatile liquids such as menthol, methyl paraben, essential oils etc.

Typical product examples are known under the names “SOS Kinder-Fieber-Pflaster” from Dr. Kleine Pharma GmbH, Germany, “Perskindol Cool Patch” or “Sportusal Cool Patch”.

A medical wound dressing is provided on the proximal side of the cooling gel layer having a smaller area than the cooling gel layer. The wound dressing therefore only covers a part of the cooling gel layer and is therefore smaller than the cooling gel layer. The wound dressing can be directly or indirectly connected with the cooling gel layer. The medical wound dressing consists of an absorbent material that can absorb moisture, in particular wound secretions and blood. The wound dressing may also contain substances which have a disinfectant and/or other medical effect on the wound.

A medical cooling pad is therefore provided that can also be used for skin injuries. The cooling pad is placed on the skin wound in such a way that the wound dressing comes to rest over or on the skin injury. The cooling gel layer of the cooling pad at the same time cools the entire zone around the skin injury. The cooling gel layer does not, however, lie directly on the skin wound. The skin injury can be treated and a skin coolant can be applied with one single action.

The medical wound dressing can, for example, be completely surrounded laterally by the cooling gel layer, thus virtually forming an island floating on the cooling gel layer. This provides that the skin injury zone in question is cooled over its entire circumference by the cooling gel layer after the application of the cooling pad.

In an embodiment of the present invention, the wound dressing can, for example, be supported by a separate adhesive tape whose area is smaller than the area of the cooling gel layer and is larger than the area of the wound dressing. The adhesive tape has an adhesive layer on its proximal side which is partially covered by the medical wound dressing. The wound dressing adheres directly to the adhesive tape in this area. The medical wound dressing and the adhesive tape thus form a classic adhesive plaster, the distal side of which is attached to the cooling gel layer.

The cooling gel layer can, for example, be self-adhesive with respect to human skin. The medical cooling pad can therefore be easily applied to the skin injury in question and adheres there without a bandage or additional adhesive.

Typical product examples therefor are known under the names “SOS Kinder-Fiber-Pflaster” from Dr. Kleine Pharma GmbH, Germany, “Perskindol Cool Patch” or “Sportusal Cool Patch”.

The adhesive layer of the adhesive tape on the skin can, for example, have a greater adhesive force than the cooling gel layer on the distal side of the tape. After the cooling effect of the cooling gel layer has been exhausted, the pad support including the cooling gel layer non-detachably attached thereto can be removed from the skin. Since the adhesive force of the adhesive layer of the adhesive tape on the skin is greater than the adhesive force between the cooling gel layer and the adhesive tape, the applied adhesive tape including the wound dressing remains on the skin during and after removal of the pad support from the skin. The adhesive plaster therefore remains on the wound after removal of the part responsible for the cooling effect so that the wound does not then need to be newly treated.

At least one removable cooling pad protective strip can, for example, be provided proximal to the wound dressing which directly covers and protects the entire exposed cooling gel layer. The cooling pad protective strip protects the cooling gel layer from drying out and protects the medical wound dressing from contamination. The cooling pad protective strip is completely removed from the proximal side of the cooling pad immediately before application of the medical cooling pad.

A cooling pad with a protective strip is also known from the above mentioned product examples.

A wound dressing protective strip may be provided as an alternative or in addition to the cooling pad protective strip directly covering and protecting only the wound dressing and, if present, the adhesive layer side of the adhesive tape. The wound dressing protective strip does not, however, cover the cooling gel layer lateral to the wound dressing or the adhesive tape. The wound dressing protective strip in particular protects the wound dressing and, if present, the adhesive layer of the adhesive tape against contamination.

The protective strips are characterized by the fact that they define only a relatively weak adhesive force between them and the cooling gel layer and the adhesive layer of the adhesive tape, so that the cooling pad protective strip and/or the wound dressing protective strip can be easily removed.

The cooling gel layer can, for example, be designed for passive cooling. The cooling gel layer contains substances with a high heat capacity and/or substances which can generate a certain evaporative cooling therefor. Particularly suitable therefor are water-binding substances that bind water or moisture.

In an embodiment of the present invention, the pad support can, for example, be provided with a layer of a carrier fabric. The function of the pad support is mainly the mechanical fixation of the cooling gel layer.

In an embodiment of the present invention, the pad support can, for example, be provided with a waterproof coating distally. This in particular prevents the cooling gel layer from drying out before and during use.

The medical cooling pad can also be individually and sterilely packaged in a bag to provide sterility until the bag is opened.

An example of the present invention is explained in greater detail below under reference to the drawings.

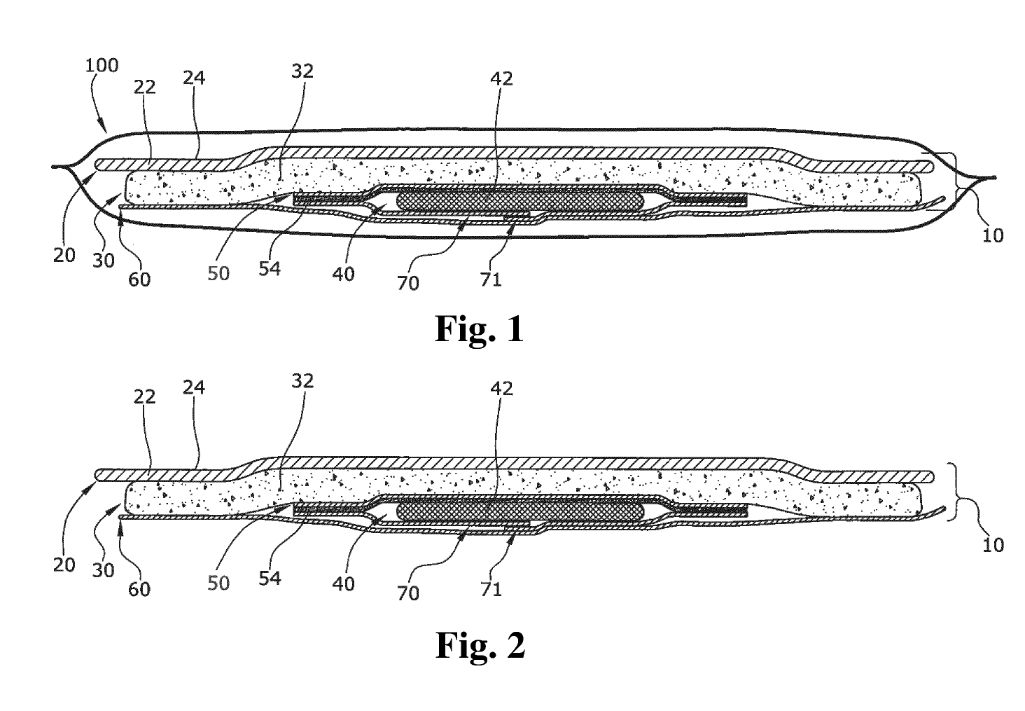

FIG. 1 shows a longitudinal section of a medical cooling pad 10 packed in a protective bag 100. The medical cooling pad 10 is used for wound care and cooling of a minor skin injury.

The medical cooling pad 10 has a multi-layered structure. From distal to proximal, the medical cooling pad 10 comprises a flexible pad support 20, which captively carries a flexible cooling gel layer 30, on which a smaller adhesive tape 50 with respect to its area adheres in the middle with the adhesive-free side, which holds a wound dressing 40 approximately in the middle of its proximal adhesive side, wherein the adhesive layer 54 of the adhesive tape 50 and the wound dressing 40 are covered by two centrally overlapping peelable wound dressing protective strips 70, 71, and wherein the proximal side of the flexible cooling gel layer 30 and the wound dressing protective strips 70, 71 are covered by a single large-area cooling pad protective strip 60.

The flexible pad support 20 essentially consists of a fleece-like carrier fabric 22 which is covered distally by a waterproof coating 24. The waterproof coating 24 consists, for example, of a suitable plastic film.

The carrier fabric 22 can be a fleece material which gives sufficient mechanical stability to the composite of flexible pad support 20 and flexible cooling gel layer 30, but which can adapt to the surface shape of the skin area in question.

The flexible cooling gel layer 30 consists of a cooling gel body 32 which is provided, for example, by a medical cooling gel. The flexible cooling gel layer 30 is relatively thick and can, for example, have a thickness of up to several millimeters. Water, methanol or moisture can be stored in a corresponding matrix within the cooling gel body 32, and other volatile liquids can also be stored alternatively or additionally.

The flexible cooling gel layer 30 is self-adhesive, i.e., it has an adhesive coating or a self-adhesive property that provides a certain adhesion of the flexible cooling gel layer 30 or the medical cooling pad 10 to the skin. The adhesive property of the flexible cooling gel layer 30 also provides that the adhesive tape 50 is held in position centrally.

The adhesive tape 50 can be a standard medical adhesive tape made of a suitable plastic film or fabric, the distal side of which is non-adhesive and the proximal side of which has the adhesive layer 54. The longitudinal and transverse dimensions of the adhesive tape 50 are smaller than the longitudinal and transverse dimensions of the flexible cooling gel layer 30.

The wound dressing 40, which is provided by an absorbent, i.e., a moisture-absorbing medical fleece 42 or tissue containing aseptic substances, is glued centrally onto the adhesive tape 50. The adhesive tape 50 along with the wound dressing 40 provide a conventional adhesive plaster 55, as it is known, for example, under the name “Hansaplast Universal Pflaster Strips”.

The proximal side of the adhesive tape 50 and/or its adhesive layer 54 as well as the wound dressing 40 are covered by two overlapping wound dressing protective strips 70, 71 made of plastic film, the two overlapping wound dressing protective strips 70, 71 developing only a relatively small adhesive force with the adhesive layer 54, i.e., the two overlapping wound dressing protective strips 70, 71 are easily removable from the adhesive layer 54.

The medical cooling pad 10 lastly comprises a full-surface cooling pad protective strip 60 made of plastic film at its proximal end, the cooling pad protective strip 60 directly covering the full surface of the flexible cooling gel layer 30 and protecting it and directly covering the two wound dressing protective strips 70, 71 centrally.

The composite of the flexible pad support 20, the self-adhesive flexible cooling gel layer 30 and the cooling pad protective strip 60 can be obtained without adhesive plaster under the product names “SOS Kinder-Fieber-Pflaster” from Dr. Kleine Pharma GmbH, Germany, “Perskindol Cool Patch” or “Sportusal Cool Patch”.

The preparation or application of the medical cooling pad 10 is shown sequentially in the FIGS. 1-6.

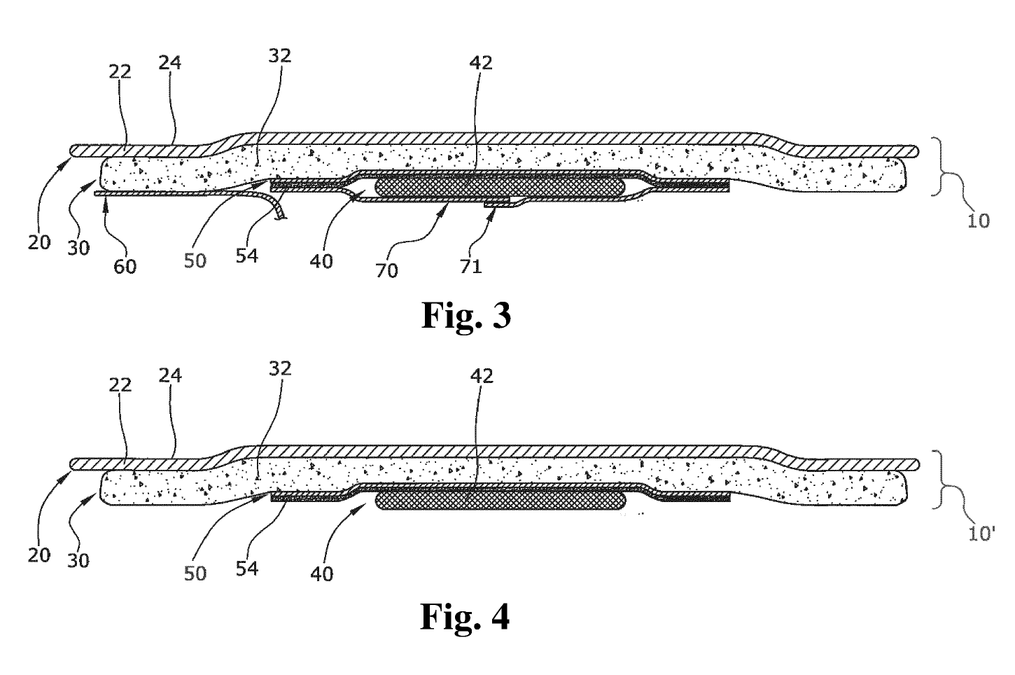

The protective bag 100, which provides the sterility of the medical cooling pad 10, is first torn open and the medical cooling pad 10 is removed from the torn protective bag 100 so that the medical cooling pad 10 is in the initial condition shown in FIG. 2. As shown in FIG. 3, the cooling pad protective strip 60 is then completely removed from the medical cooling pad 10. Finally, the now accessible two wound dressing protective strips 70, 71 are completely removed from the adhesive tape 50 so that, as shown in FIG. 4, the medical cooling pad 10′ is now ready for application to the skin.

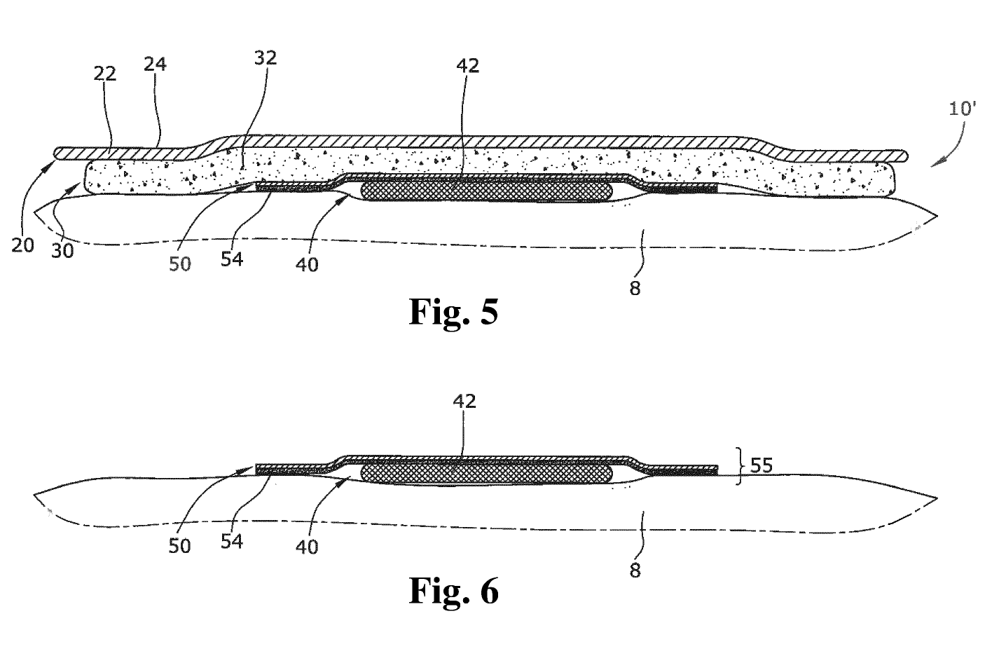

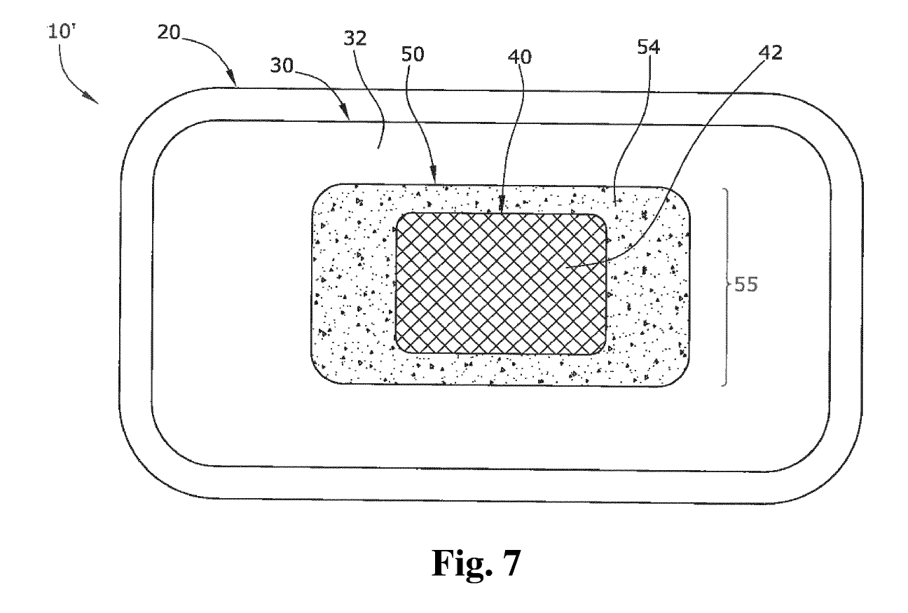

The condition of the medical cooling pad 10′ when applied to the skin 8 is shown in FIG. 5. It is here already recognizable that, in the area of the adhesive tape 50, the adhesive layer 54 of the adhesive tape 50 adheres directly to the skin 8. The self-adhesive flexible cooling gel layer 30 adheres directly to the skin 8 in all areas not directly covered by the adhesive tape 50.

As soon as the cooling effect of the flexible cooling gel layer 30 is exhausted, the flexible pad support 20 including the flexible cooling gel layer 30 fixed thereto can be removed from the skin 8. Since the adhesive force between the flexible cooling gel layer 30 and the distal non-adhesive side of the adhesive tape 50 is relatively small, the adhesive force between the adhesive layer 54 and the skin 8 is sufficient for the adhesive tape 50, including the wound dressing 40, to remain attached to the skin 8 when the composite of flexible pad support 20 and flexible cooling gel layer 30 is removed. More air reaches the skin 8 after removal of the flexible cooling gel layer 30, which promotes the healing process.

The present invention is not limited to embodiments described herein; reference should be had to the appended claims.

What is claimed is:

1-10. (canceled)

11. A medical cooling pad for cooling skin, the medical cooling pad comprising:
a distal flexible pad support;
a flexible cooling gel layer arranged on a proximal side of the distal flexible pad support; and
a medical wound dressing arranged on a proximal side of the flexible cooling gel layer, the medical wound dressing comprising an area which is smaller than an area of the flexible cooling gel layer.

12. The medical cooling pad as recited in claim 11, wherein the medical wound dressing is laterally completely surrounded by the flexible cooling gel layer.

13. The medical cooling pad as recited in claim 11, further comprising:
a separate adhesive tape comprising,
an adhesive layer on a proximal side, and
an area which is smaller than the area of the flexible cooling gel layer and which is larger the area of the medical wound dressing.

14. The medical cooling pad as recited in claim 13, wherein,
the adhesive layer is configured to provide an adhesive force on the skin,
the flexible cooling gel layer is configured to provide an adhesive force on the skin, and
the adhesive force of the adhesive layer on the skin is greater than the adhesive force of the flexible cooling gel layer on the skin.

15. The medical cooling pad as recited in claim 13, further comprising:
at least one removable wound dressing protective strip which is arranged proximally to the medical wound dressing, the at least one removable wound dressing protective strip being configured to directly cover and to protect the medical wound dressing and the separate adhesive tape, but to not cover the cooling gel layer.

16. The medical cooling pad as recited in claim 11, wherein the flexible cooling gel layer is configured to be self-adhesive with respect to the skin.

17. The medical cooling pad as recited in claim 11, further comprising:
at least one removable cooling pad protective strip which is arranged proximally to the medical wound dressing, the at least one removable cooling pad protective strip being arranged to directly cover and to protect the flexible cooling gel layer.

18. The medical cooling pad as recited in claim 11, further comprising:
at least one removable wound dressing protective strip which is arranged proximally to the medical wound dressing, the at least one removable wound dressing protective strip being configured to directly cover and to protect the medical wound dressing but to not cover the flexible cooling gel layer.

19. The medical cooling pad as recited in claim 11, wherein the flexible cooling gel layer is configured to provide a passive cooling.

20. The medical cooling pad as recited in claim 11, wherein the distal flexible pad support comprises at least one of a layer of a support fabric and a waterproof coating.

21. A medical cooling pad for cooling skin, the medical cooling pad comprising:
a distal flexible pad support;
a flexible cooling gel layer arranged on a proximal side of the distal flexible pad support;
a medical wound dressing arranged on a proximal side of the flexible cooling gel layer, the medical wound dressing comprising an area which is smaller than an area of the flexible cooling gel layer; and
a separate adhesive tape comprising,
an adhesive layer on a proximal side, and
an area which is larger than the area of the medical wound dressing.

22. The medical cooling pad as recited in claim 21, wherein the area of the separate adhesive tape is smaller than the area of the flexible cooling gel.

23. The medical cooling pad as recited in claim 21, wherein the medical wound dressing is laterally completely surrounded by the flexible cooling gel layer.

24. The medical cooling pad as recited in claim 21, wherein the flexible cooling gel layer is configured to be self-adhesive with respect to the skin.

25. The medical cooling pad as recited in claim 21, wherein,
the adhesive layer is configured to provide an adhesive force on the skin,
the flexible cooling gel layer is configured to provide an adhesive force on the skin, and
the adhesive force of the adhesive layer on the skin is greater than the adhesive force of the flexible cooling gel layer on the skin.

26. The medical cooling pad as recited in claim 21, further comprising:
at least one removable cooling pad protective strip which is arranged proximally to the medical wound dressing, the at least one removable cooling pad protective strip being arranged to directly cover and to protect the flexible cooling gel layer.

27. The medical cooling pad as recited in claim 21, further comprising:
at least one removable wound dressing protective strip which is arranged proximally to the medical wound dressing, the at least one removable wound dressing protective strip being configured to directly cover and to protect the medical wound dressing and the separate adhesive tape, but to not cover the cooling gel layer.

28. The medical cooling pad as recited in claim 21, wherein the flexible cooling gel layer is configured to provide a passive cooling.

29. The medical cooling pad as recited in claim 21, wherein the distal flexible pad support comprises at least one of a layer of a support fabric and a waterproof coating.
